(19) United States
(12) Patent Application Publication
(10) Pub. No.: US 2024/0277361 A1
(43) Pub. Date: Aug. 22, 2024

(54) ASPIRATION CATHETER HAVING A BRAIDED MARKER BAND AND IMPROVED VISIBILITY UNDER FLUOROSCOPY

(71) Applicant: DePuy Synthes Products, Inc., Raynham, MA (US)

(72) Inventors: Pedro PEDROSO, Raynham, MA (US); Chadwin HANNA, Raynham, MA (US)

(73) Assignee: DePuy Synthes Products, Inc., Raynham, MA (US)

(21) Appl. No.: 18/582,099

(22) Filed: Feb. 20, 2024

Related U.S. Application Data
(60) Provisional application No. 63/447,523, filed on Feb. 22, 2023.

(51) Int. Cl. A61B 17/22; A61B 17/00; A61L 29/02; A61M 25/00

(52) U.S. Cl. CPC A61B 17/22; A61B 17/00234; A61B 2017/00336; A61L 29/02; A61M 25/0012; A61M 25/0053

(57) ABSTRACT

A catheter is disclosed for treating aneurysms. The catheter can include a liner having a proximal end, a distal end, an outer surface, and an internal lumen extending from the proximal end to the distal end along a longitudinal axis A. A braid is disposed on the outer surface of the liner and extends from the proximal end to the distal end of the liner. The braid comprises a plurality of wires wherein a first portion of the wires are made from tungsten and a second portion of the wires are made from a different material. A first distal section of the braid has a pic rate of about 330 picks per inch (“ppi”). A jacket is disposed about the braid and extends from the proximal end to the distal end.

### CROSS-REFERENCE TO RELATED APPLICATIONS

This application claims the benefit of U.S. Provisional Application No. 63/447,523 filed Feb. 22, 2023, the disclosure of which is incorporated herein by reference in its entirety.

### FIELD OF DISCLOSURE

The present disclosure generally relates devices and methods for removing acute blockages from blood vessels during intravascular medical treatments. More specifically, the present disclosure relates to aspiration retrieval catheters into which an object or objects can be retrieved.

### BACKGROUND

Clot retrieval aspiration catheters and devices are used in mechanical thrombectomy for endovascular intervention, often in cases where patients are suffering from conditions such as acute ischemic stroke (AIS), myocardial infarction (MI), and pulmonary embolism (PE). Accessing the neurovascular bed is challenging with conventional technology, as the target vessels are small in diameter, remote relative to the site of insertion, and highly tortuous. These catheters are frequently of great length and must follow the configuration of the blood vessels in respect of all branching and windings. Traditional devices are often either too large in profile, lack the deliverability and flexibility needed to navigate particularly tortuous vessels, or are not effective at removing a clot when delivered to the target site.

As a result, there remains a need for improved catheter designs that refine current methods for improving the lateral flexibility of the catheter to improve the trackability of the catheter. The presently disclosed designs are aimed at providing an improved catheter having improved deliverability and flexibility needed to navigate particularly tortuous vessels. The presently disclosed designs also provide a catheter having a braided metal wire reinforcing layer capable of improving visibility of the catheter under fluoroscopy, while still being flexible, including at the distal end of the catheter, to improve the trackability of the catheter as it navigates tortuous vessels.

For vascular catheters to reach their target site a catheter is made such that, at its proximal end, the catheter is designed to be relatively stiff and gradually becomes less stiff/more flexible at the catheter distal end. This is mainly achieved by using harder materials proximally (Rockwell D from 180 to 90) and softer materials distally (Rockwell≤35D to Shore 72A to 30A) for the outer jacket materials. Vascular catheters are navigated within vessels, such as, for example, in the human anatomy, with the aid of fluoroscopic imaging which utilizes x-rays. However, the polymers of catheters do not attenuate x-ray very well as the catheter is not visible under fluoroscopy. Any metal components of the catheter body, for example, a braided or coiled wire reinforcement layer, are not significantly radiopaque. Stainless steel grades, such as, for example, 304, 316, 404, etc. are often used as the braid material. However, stainless steels are moderately radiopaque but not to the level clinically acceptable to behave as a marker band. For this reason, conventional catheters incorporate a highly x-ray attenuating metal marker, called a marker band, at the very distal tip of the catheter. FIG. 9 shows how a conventional catheter appears under fluoroscopy. Only the distal tip of conventional catheters is visible under fluoroscopy, because of the marker band, and this only reveals where the distal tip of the catheter resides in the vasculature. Thus, medical practitioners rely only on physical feedback from conventional catheter designs to aide in their navigation of such devices within the vasculature.

For most catheter designs the metal of choice is a stainless-steel grade (304, 316, 404, etc.). Stainless-steels are moderately radiopaque but not to the level that is clinically acceptable to function as a marker band. These braid wires could be made from more radiopaque materials such as platinum, however platinum does not resist deformation very well. Catheters having platinum braids are not very springy to recover to their original shape after being deformed and are instead rather easily plastically deformed. Therefore, catheters having platinum braids do not have the mechanical structure to be used as in vascular catheters.

### SUMMARY

The catheter in accordance with the present disclosure, allows visualization of the entire length of the catheter. The catheter allows a medical practitioner to adjust their navigation technique with visual cues. Therefore, as all regions of the catheter traverse anatomy, a medical practitioner can adjust the way she or he pushes or pulls the catheter and accessories based on visible feedback of the entire length of the catheter which permits visualization of how the shaft is bending at any point along the radiopaque shaft of the catheter.

Because the entire length of the catheter in accordance with the present disclosure, is visible under fluoroscopy, there is no need for a separate ridged marker band at the distal end of the catheter. The distal tip of the catheter in accordance with the present disclosure is radiopaque due to a compression of the braid at the distal end of the catheter. This structure allows for a radiopaque tip that is also flexible at the distal end of the catheter. Thus, the catheter in accordance with the present disclosure provides a better and more seamless navigation through tortuous anatomy. The distal end of the catheter in accordance with the present disclosure does not include a separate radiopaque marker band but instead relies on a continuous section of the braid component itself to provide a catheter design. Thus, the catheter in accordance with the present disclosure, allows visualization of the entire length of the catheter and even greater visualization at the distal end without the need for a separate, rigid marker band. In some examples, the rigid marker band of a conventional catheter can become detached from the rest of the catheter and remain in the vasculature during use of the catheter.

In some examples, a catheter in accordance with the present disclosure is disclosed for treating aneurysms. The catheter can include a liner having a proximal end, a distal end, an outer surface, and an internal lumen extending from the proximal end to the distal end along a longitudinal axis A. A braid is disposed on the outer surface of the liner and extends from the proximal end to the distal end of the liner. The braid comprises a plurality of wires wherein a first portion of the wires are made from tungsten and a second portion of the wires are made from a different material. A jacket is disposed about the braid and extends from the proximal end to the distal end.

### BRIEF DESCRIPTION OF THE DRAWINGS

The above and further aspects of this disclosure are further discussed with reference to the following description in conjunction with the accompanying drawings, in which like numerals indicate like structural elements and features in various figures. The drawings are not necessarily to scale, emphasis instead being placed upon illustrating principles of the disclosure. The figures depict one or more implementations of the inventive devices, by way of example only, not by way of limitation.

FIG. 1 is a perspective view of a catheter in accordance with the present disclosure;

FIG. 2 is a top view of the catheter in accordance with the present disclosure with a core shown in use during manufacture;

FIG. 3 is a partial exploded view, with parts broken away, showing an outer jacket segment of an outer cover, which is about a metal braid, which is about an inner liner;

FIG. 4 is a view of the metal braid of the catheter with different segments of braid density;

FIG. 5 is a top view of the catheter with parts broken away;

FIG. 6A is a partial top view of a prior art catheter, with the outer jacket not shown;

FIG. 6B is a cross-sectional view from section 6B of FIG. 6A, showing the hollow one-piece marker band at the distal end of the catheter;

FIG. 7 is a partial top view of a distal end of the catheter in accordance with the present disclosure, with the outer jacket not shown;

FIGS. 8A and 8B are pictures of a catheter in accordance with the present disclosure shown within a vessel; and

FIG. 9 is a picture of a catheter in accordance with the prior art shown within a vessel.

### DETAILED DESCRIPTION

The following detailed description is merely exemplary in nature and is not intended to limit the disclosure or the application and uses of the disclosure. Although the description of the disclosure is in many cases in the context of treatment of intracranial arteries, the disclosure may also be used in other body passageways as previously described.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g. “about 80%” may refer to the range of values from 61% to 99%.

As discussed herein, a “patient” or “subject” can be a human or any animal. It should be appreciated that an animal can be a variety of any applicable type, including, but not limited to, mammal, veterinarian animal, livestock animal or pet-type animal, etc. As an example, the animal can be a laboratory animal specifically selected to have certain characteristics similar to a human e.g., rat, dog, pig, monkey, or the like.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

It must also be noted that, as used in the specification and the appended claims, the singular forms “a,” “an,” and “the” include plural referents unless the context clearly dictates otherwise. Ranges can be expressed herein as from “about” or “approximately” one particular value and/or to “about” or “approximately” another particular value. When such a range is expressed, other exemplary examples include from the one particular value and/or to the other particular value.

Accessing cerebral, coronary and pulmonary vessels involves the use of a number of commercially available products and conventional procedural steps. Access products such as guidewires, guide catheters, angiographic catheters and microcatheters are described elsewhere and are regularly used in catheter lab procedures. It is assumed in the descriptions below that these products and methods are employed in conjunction with the device and methods of this disclosure and do not need to be described in detail.

Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

A catheter in accordance with the present disclosure includes an inner lumen liner comprising a first polymer material, a braided metal wire jacket comprising a plurality of metal wires, and an outer liner comprising a second polymer material that may be the same or different from the first polymer material. A portion of the plurality of metal wires are made of tungsten and a portion of the plurality of metal wires are made of stainless steel. Tungsten is visible under fluoroscopy and provides a user with visual feedback of the entire catheter length when tracking a catheter in the vasculature. In addition, the braid is made with a higher pic count adjacent to the distal end to provide increased visibility at the distal end but without the disadvantages of using a rigid marker band.

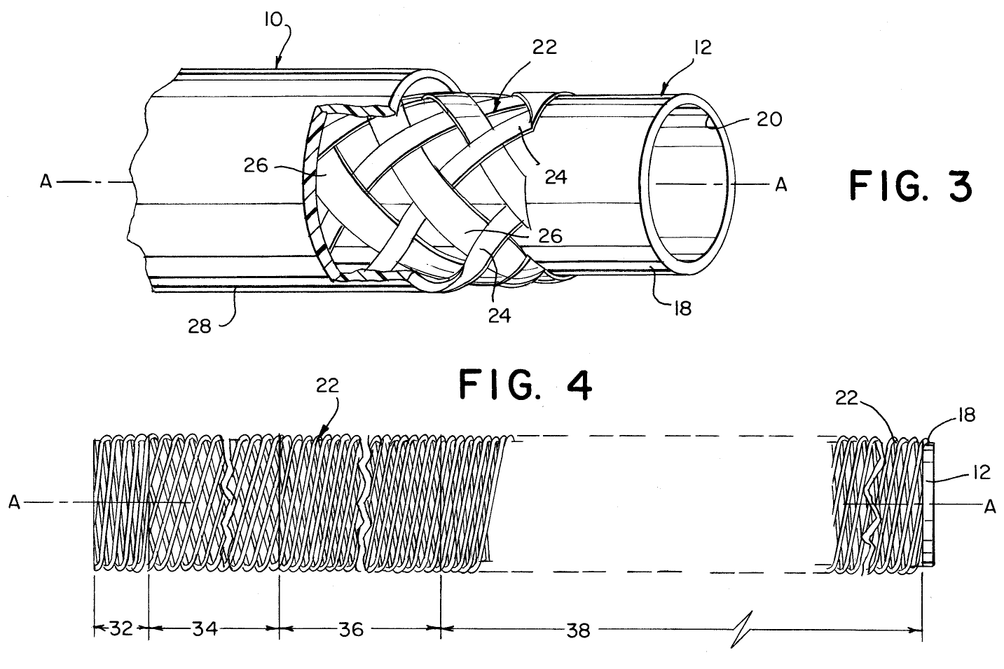

Referring now to FIGS. 1-5, and 7, a catheter 10 for navigating within a body lumen. Catheter 10 includes a core or liner 12, a braid 22, and an outer jacket 28. Catheter 10 has a proximal end 14 and a distal end 16. Core 12 has an outer surface 18, an internal lumen 20 extending from the proximal end 14 to the distal end 16, and a longitudinal axis A. Braid 22 is disposed on the outer surface 18 of liner 12 and extends from the proximal end 14 to the distal end 16 of liner 12. Braid 22 comprises a plurality of wires 24, 26. A first portion of the wires 24 are made from tungsten and a second portion of the wires 26 are made from a different material, such as, for example, stainless steel. The tungsten wires 24 can be made from tungsten and its alloys, such as, for example, WRe, WCr, WTa, WMo, WTi. The stainless steel wires can be made from, for example, stainless steel grades 304, 304V, 316, and 404. Jacket 28 is disposed about braid 22 and extends from the proximal end 14 to the distal end 16.

Refractory metal/alloys such as Tungsten (W), MoRe, NbHf, NbW, VTa, or more generally Period 6 metals from the periodic table are very radiopaque. Many of these alloys are so hard that when incorporated into a braid they result in a mechanically strong metal backbone that has a very elastic or springy properties. That is, a catheter having braid wires, of which at least a portion of the braid wires are made from a Period 6 metal resists radial loads when in configured in a tubular braid. The catheter in accordance with the present disclosure incorporates such a metal as some of the braided wires to achieve clinically relevant radiopacity. This increase in radiopacity is further achieved by compressing the braid as much as the braid wire diameters will allow in a distal most section of the braid to form a braided marker-band.

The first portion of wires 24 is about half of the total number of wires and the second portion of the wires 26 is also about half of the total number of wires. In one example of the disclosure, the first portion of the wires 24 comprises eight wires and the second portion of the wires 26 comprises eight wires.

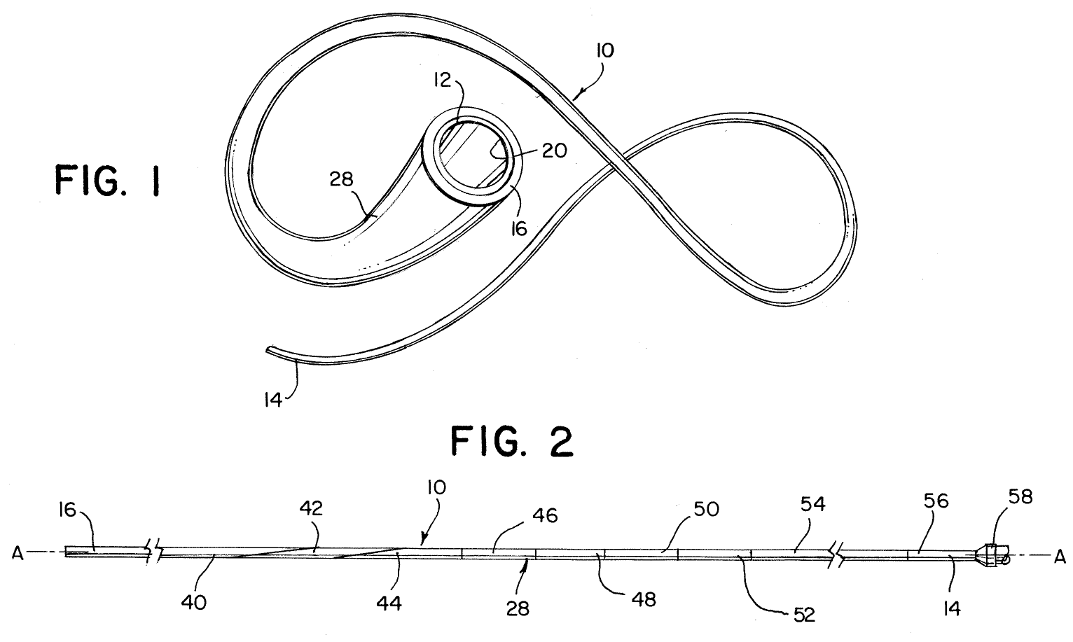

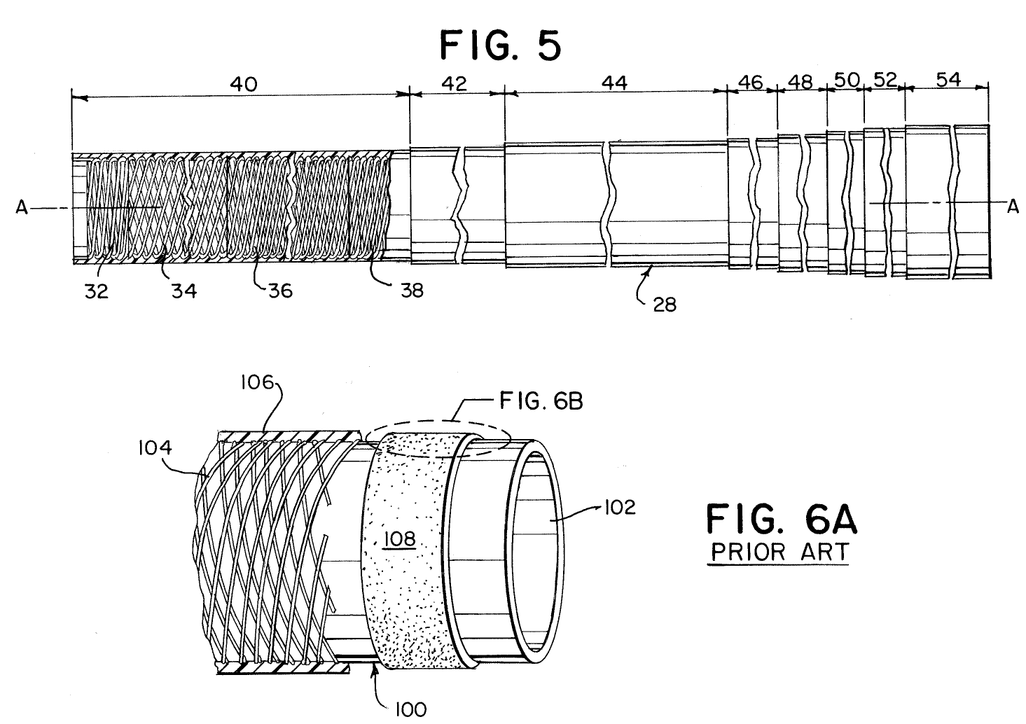

Liner 12 is made of PTFE in one example of the present disclosure. Jacket 28 is made of a polymer material in one example of the present disclosure. As shown in FIGS. 2 and 5, jacket 28 is comprised of a plurality of segments 40, 42, 44, 46, 48, 50, 52, and 54. In one example, eight jackets together combine to make up jacket 28. A strain relief section 56 is connected to segment 54 at its proximal end. Strain relief section 56 connects to a hub luer 58 as is known in the art.

Braid 22 has a plurality of different pic rates along the longitudinal axis A of braid 22. In one example, braid 22 has at least four axial sections 32, 34, 36, 38 extending from the distal end 16 to the proximal end 14. The at least four axial sections include a first most distal section 32, a second distal section 34 adjacent to the first most distal section, a third distal section 36 adjacent to the second distal section, and a fourth proximal most section 38 adjacent to the third distal section 36. The second distal section 34 being between the first distal section 32 and the third distal section 38. The third distal section 36 being between the second distal section 34 and the fourth distal section 38. The fourth most proximal section 38 being adjacent to the third distal section 36.

Each braid section 32, 34, 36, 38 has a different pic rate. Braid first distal most section 32 has a pic rate of about 330 picks per inch (“ppi”). Braid first distal section 32 has an axial length of about 0.010 inches to about 0.039 inches. Braid second distal section 34 has a pic rate of about 105 ppi. Braid second distal section 34 has an axial length of about 7.87 inches. Braid third distal section 36 has a variable pic rate of about 105 ppi to about 195 ppi. Braid third distal section 36 has a variable pic rate of about 105 ppi adjacent to a proximal end of the third distal section to about 195 ppi adjacent to a distal end of the third distal section. Braid third distal section 36 has a variable pic rate that transitions continuously from the proximal end of the third distal section to the distal end of the third distal section. Braid third distal section 36 has an axial length of about 7.87 inches. Braid fourth proximal section 38 has a pic rate of about 195 ppi. Braid fourth proximal section 38 has an axial length of about 44.0 inches.

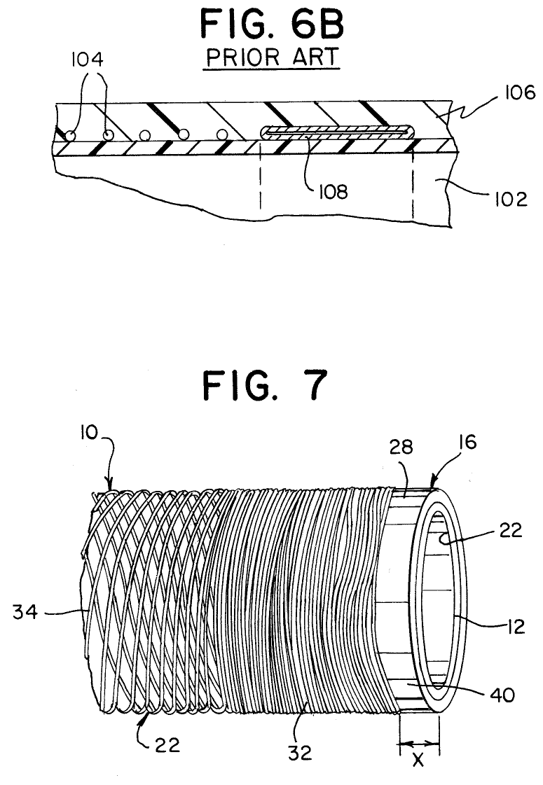

Referring now to FIG. 7, the distal end 16 of catheter 10 is shown. First braid section 32 is shown and only a portion of outer jacket 28 is shown so that braid 22 sections 32 and 34 are visible. The distal end of the braid's distal most section 32 is spaced from the distal end of the catheter by distance X. The distal end of the catheter, within distance X, is comprised of core 12 and outer jacket 28, specifically segment 40 of outer jacket 28, and no braid reinforcement. The distal section of braid 22 includes first braid section 32. Because the distal section of catheter 10 does not have a rigid marker band, but has a flexible braid section 31, the distal end 16 of catheter 10 is relatively flexible and atraumatic. Distance X is about 0.025 cm to about 0.050 cm, or about 0.010 inches to about 0.020 inches.

Catheter 10 has in an example of the present disclosure an inner diameter of about 0.092 inches from its proximal end 14 to its distal end 16. Catheter 10 also has, in an example of the present disclosure, an outer diameter that tapers from an outer diameter of about 0.108 inches at proximal end 14 down to about 0.101 inches at distal end 16. Catheter 10, in an example of the present disclosure, can have various working lengths, such as, for example, of about 114 cm, or about 122 cm, or about 130 cm, or about 135 cm. Catheter 10 can also have a hydrophilic coating on its inner diameter for a length of about 50 cm extending from distal end 16 toward the proximal end.

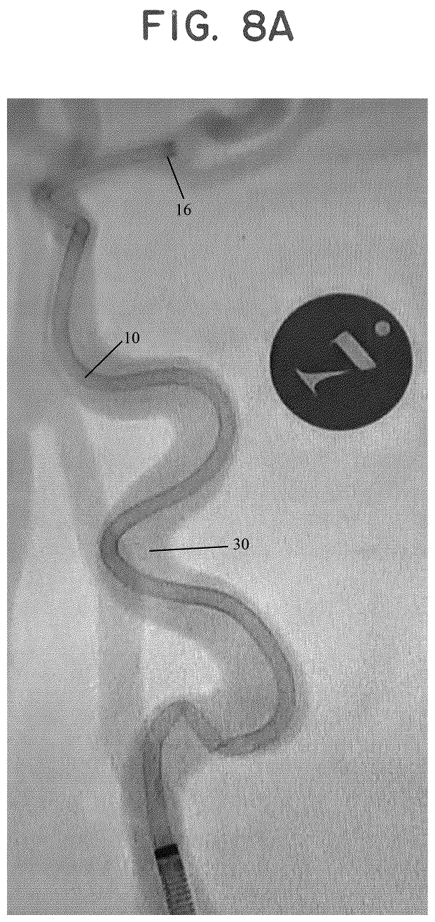

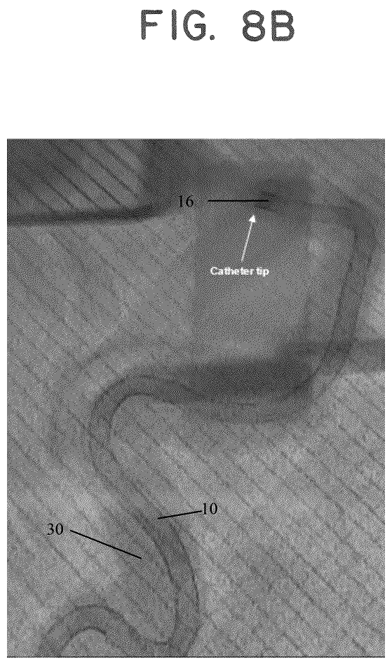

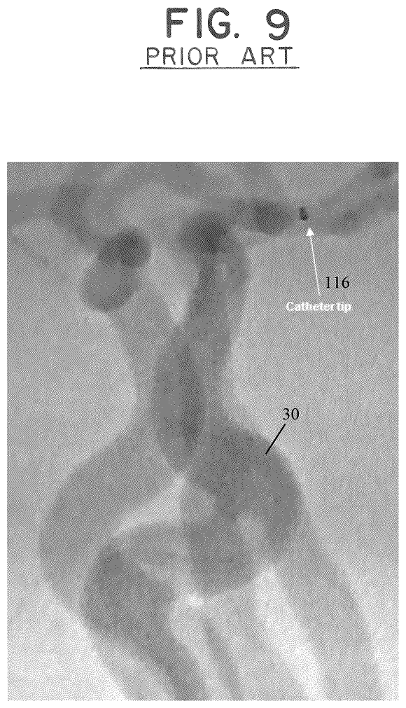

Referring now to FIGS. 8A and 8B, a picture of a catheter 10 in accordance with the present disclosure is shown under fluoroscopy within a vessel 30. As can be seen, the distal end 16 of the catheter is visible due to the increased pic rate of braid 22 at the first most distal section 32 of braid 22. Also, the outline of the entire length of catheter 10 can be seen under fluoroscopy due to braid 22 being partially comprised of tungsten wires. The vessels 30 within the vasculature can be seen as a hazy shade in FIGS. 8A, 8B and 9. The circle labeled 7 in FIG. 8A is shown for relative size. Circle 7 has a diameter of about 0.949 inches. FIG. 9 shows a conventional catheter under fluoroscopy within a vessel 30. The conventional catheter cannot be seen in FIG. 9, but the distal end 116 of the conventional catheter can be seen.

Referring now to FIGS. 6A and 6B, the distal end of a prior art catheter 100 is illustrated. Catheter 100 has a core 102, a braid 104 about the core and a jacket 106 about the braid. Catheter 100 also has a marker band 108 at the distal end of catheter and embedded between the core 102 and jacket 106. Marker band 108 is made of a material that is radiopaque under fluoroscopy, such as, for example, platinum/iridium, tantalum or gold and usually has an axial length of between about 0.5 mm and about 2 mm. As shown in FIGS. 6A and 6B, marker band 108 is a rigid one-piece hollow annular band disposed about core 102 and radially within jacket 106. As such, marker band 108, which is located at the distal end of the catheter, is not flexible. Because marker band 108 is not flexible, catheter 100 can have issues when tracking because of the use of a rigid marker band 108 at the distal end of the catheter. Rigid marker band 108 adversely affects catheter navigation since the metal hypotube marker-band 108 is ridged and cannot flex. In addition, traditional marker bands must be separately affixed to the catheter shaft. These conventional marker bands have been known to detach from the catheter body during clinical use, especially if the gap between the marker-band and the rest of the catheter body is too large.

Aspects of the disclosure are also provided by the following numbered clauses:
Clause 1: A catheter 10 for navigating within body lumen, the catheter comprising:
a liner 12 having a proximal end 14, a distal end 16, an outer surface 18, an internal lumen 20 extending from the proximal end 14 to the distal end 16, and a longitudinal axis A;
a braid 22 disposed on the outer surface 18 of the liner 12 and extending from the proximal end 14 to the distal end 16 of the liner 12, the braid 22 being comprises a plurality of wires 24, 26, wherein a first portion of the wires 24 are made from tungsten and a second portion of the wires 26 are made from a different material; and
a jacket 28 disposed about the braid 22 and extending from the proximal end 14 to the distal end 16.
Clause 2. The catheter of clause 1, wherein the first portion of the wires 24 is about half of the wires and the second portion of the wires 26 is about half of the wires.
Clause 3. The catheter of clause 1 or 2, wherein the first portion of the wires 24 is half of the wires and the second portion of the wires 26 is half of the wires.
Clause 4. The catheter of any of clauses 1-3, wherein the first portion of the wires 24 comprises eight wires and the second portion of the wires 26 comprises eight wires.
Clause 5. The catheter of any of clauses 1-4, wherein the second portion of the wires 26 are made from stainless steel.
Clause 6. The catheter of any of clauses 1-5, wherein the liner 12 is made of PTFE.
Clause 7. The catheter of any of clauses 1-6, wherein the jacket 28 is made of a polymer material.
Clause 8. The catheter of any of clauses 1-7, wherein the jacket 28 is comprised of a plurality of segments.
Clause 9. The catheter of any of clauses 1-8, wherein the jacket 28 is comprised of eight segments.
Clause 10. The catheter of any of clauses 1-9, wherein the braid 22 has a plurality of pic rates along the longitudinal axis.
Clause 11. The catheter of any of clauses 1-10, wherein the braid has at least four axial sections extending from the distal end 16 towards the proximal end 14, the at least five axial sections includes a first most distal section 32, a second distal section 34 adjacent to the first most distal section, a third distal section 36 adjacent to the second distal section, and a fourth proximal section 38 adjacent to the third distal section, the second distal section being between the first distal section and the third distal section, the third distal section being between the second distal section and the fourth proximal section.
Clause 12. The catheter of any of clauses 1-11, wherein the braid first distal section 32 has a pic rate of about 330 picks per inch (“ppi”).
Clause 13. The catheter of any of clauses 1-12, wherein the braid first distal section 32 has an axial length of about 0.010 inches to about 0.039 inches.
Clause 14. The catheter of any of clauses 1-13, wherein the braid second distal section 34 has a pic rate of about 105 ppi.
Clause 15. The catheter of any of clauses 1-14, wherein the braid second distal section 34 has an axial length of about 7.87 in.
Clause 16. The catheter of any of clauses 1-15, wherein the braid third distal section 36 has a variable pic rate of about 105 ppi to about 195 ppi.
Clause 17. The catheter of any of clauses 1-16, wherein the braid third distal section 36 has a variable pic rate of about 105 ppi adjacent to a proximal end of the third distal section to about 195 ppi adjacent to a distal end of the third distal section.
Clause 18. The catheter of any of clauses 1-17, wherein the braid third distal section 36 has a variable pic rate that transitions continuously from the proximal end of the third distal section to the distal end of the third distal section.
Clause 19. The catheter of any of clauses 1-16, wherein the braid third distal section 36 has an axial length of about 7.87 in.
Clause 20. The catheter of any of clauses 1-19, wherein the braid fourth proximal section 38 has a pic rate of about 195 ppi.
Clause 21. The catheter of any of clauses 1-20, wherein the braid fourth proximal section 38 has an axial length of about 44.0 in.
Clause 22. A catheter 10 for navigating within body lumen, the catheter comprising: a liner 12 having a proximal end 14, a distal end 16, an outer surface 18, an internal lumen 20 extending from the proximal end 14 to the distal end 16, and a longitudinal axis A;
a braid 22 disposed on the outer surface 18 of the liner 12 and extending from the proximal end 14 to the distal end 16 of the liner 12, the braid 22 has at least four axial sections extending from the distal end 16 towards the proximal end 14, the at least four axial sections includes a first most distal section 32, a second distal section 34 adjacent to the first most distal section 32, a third distal section 36 adjacent to the second distal section 34, and a fourth proximal section 38 adjacent to the third distal section 36, the second distal section 34 being between the first distal section 32 and the third distal section 36, the third distal section 36 being between the second distal section 34 and the fourth proximal section 38, the fourth proximal section 38 being adjacent to and proximal to the third distal section 36, the braid first distal section 32 having a pic rate of about 330 picks per inch (“ppi”); and
a jacket 28 disposed about the braid 22 and extending from the proximal end 14 to the distal end 16.
Clause 23. The catheter of clause 22, wherein the braid first distal section 32 has an axial length of about 0.010 inches to about 0.039 inches.
Clause 24. The catheter of any of clauses 22-23, wherein the braid second distal section 34 has a pic rate of about 105 ppi.
Clause 25. The catheter of any of clauses 22-24, wherein the braid second distal section 34 has an axial length of about 7.87 in.
Clause 26. The catheter of any of clauses 22-25, wherein the braid third distal section 36 has a variable pic rate of about 105 ppi to about 195 ppi.
Clause 27. The catheter of any of clauses 22-26, wherein the braid third distal section 36 has a variable pic rate of about 105 ppi adjacent to a proximal end of the third distal 36 section to about 195 ppi adjacent to a distal end of the third distal section 36.
Clause 28. The catheter of any of clauses 22-27, wherein the braid third distal section 36 has a variable pic rate that transitions continuously from the proximal end of the third distal section 36 to the distal end of the third distal section 36.
Clause 29. The catheter of any of clauses 22-28, wherein the braid third distal section 36 has an axial length of about 7.87 in.
Clause 30. The catheter of any of clauses 22-29, wherein the braid fourth proximal section 38 has a pic rate of about 195 ppi.
Clause 31. The catheter of any of clauses 22-30, wherein the braid fourth proximal section 38 has an axial length of about 44.0 in.
Clause 32. The catheter of any of clauses 22-31, wherein the braid 22 comprises a plurality of wires, wherein a first portion of the wires 24 are made from tungsten and a second portion of the wires 26 are made from a different material.
Clause 33. The catheter of any of clauses 22-32, wherein the first portion of the wires 24 comprises eight wires and the second portion of the wires 26 comprises eight wires.
Clause 34. The catheter of any of clauses 22-33, wherein the second portion of the wires 26 are made from stainless steel.

The descriptions contained herein are examples of embodiments of the disclosure and are not intended in any way to limit the scope of the disclosure. As described herein, the disclosure contemplates many variations and modifications of a catheter. Modifications and variations apparent to those having skilled in the pertinent art according to the teachings of this disclosure are intended to be within the scope of the claims which follow.

What is claimed is:

1. A catheter for navigating within body lumen, the catheter comprising:
a liner having a proximal end, a distal end, an outer surface, an internal lumen extending from the proximal end to the distal end, and a longitudinal axis;
a braid disposed on the outer surface of the liner and extending from the proximal end to the distal end of the liner, the braid being comprises a plurality of wires, wherein a first portion of the wires are made from tungsten and a second portion of the wires are made from a different material; and
a jacket disposed about the braid and extending from the proximal end to the distal end.

2. The catheter of claim 1, wherein the first portion of the wires is about half of the wires and the second portion of the wires is about half of the wires.

3. The catheter of claim 2, wherein the first portion of the wires is half of the wires, and the second portion of the wires is half of the wires.

4. The catheter of claim 3, wherein the first portion of the wires comprises eight wires and the second portion of the wires comprises eight wires.

5. The catheter of claim 1, wherein the braid has a plurality of pic rates along the longitudinal axis.

6. The catheter of claim 5, wherein the braid has at least four axial sections extending from the distal end towards the proximal end, the at least five axial sections includes a first most distal section, a second distal section adjacent to the first most distal section, a third distal section adjacent to the second distal section, and a fourth proximal section adjacent to the third distal section, the second distal section being between the first distal section and the third distal section, the third distal section being between the second distal section and the fourth proximal section.

7. The catheter of claim 6, wherein the braid first distal section has a pic rate of about 330 picks per inch (“ppi”).

8. The catheter of claim 7, wherein the braid first distal section has an axial length of about 0.010 inches to about 0.039 inches.

9. The catheter of claim 8, wherein the braid second distal section has a pic rate of about 105 ppi.

10. The catheter of claim 9, wherein the braid second distal section has an axial length of about 7.87 in.

11. The catheter of claim 10, wherein the braid third distal section has a variable pic rate of about 105 ppi to about 195 ppi.

12. The catheter of claim 11, wherein the braid third distal section has a variable pic rate of about 105 ppi adjacent to a proximal end of the third distal section to about 195 ppi adjacent to a distal end of the third distal section.

13. The catheter of claim 12, wherein the braid third distal section has a variable pic rate that transitions continuously from the proximal end of the third distal section to the distal end of the third distal section.

14. The catheter of claim 13, wherein the braid third distal section has an axial length of about 7.87 in.

15. The catheter of claim 14, wherein the braid fourth proximal section has a pic rate of about 195 ppi.

16. The catheter of claim 15, wherein the braid fourth proximal section has an axial length of about 44.0 in.

17. A catheter for navigating within body lumen, the catheter comprising:
a liner having a proximal end, a distal end, an outer surface, an internal lumen extending from the proximal end to the distal end, and a longitudinal axis,
a braid disposed on the outer surface of the liner and extending from the proximal end to the distal end of the liner, the braid has at least four axial sections extending from the distal end towards the proximal end, the at least four axial sections includes a first most distal section, a second distal section adjacent to the first most distal section, a third distal section adjacent to the second distal section, and a fourth proximal section adjacent to the third distal section, the second distal section being between the first distal section and the third distal section, the third distal section being between the second distal section and the fourth proximal section, the fourth proximal section being adjacent to and proximal to the third distal section, the braid first distal section having a pic rate of about 330 picks per inch (“ppi”); and
a jacket disposed about the braid and extending from the proximal end to the distal end.

18. The catheter of claim 17, wherein the braid first distal section has an axial length of about 0.010 inches to about 0.039 inches.

19. The catheter of claim 18, wherein the braid second distal section has a pic rate of about 105 ppi.

20. The catheter of claim 19, wherein the braid third distal section has a variable pic rate of about 105 ppi to about 195 ppi.
